(19) United States
(12) Patent Application Publication
(10) Pub. No.: US 2021/0346256 A1
(43) Pub. Date: Nov. 11, 2021

(54) Oral Care Compositions Comprising Tin

(71) Applicant: The Procter & Gamble Company, Cincinnati, OH (US)

(72) Inventors: Arif Ali Baig, Mason, OH (US); Aaron Reed Biesbrock, Maineville, OH (US); Ehinomen Christine Enekabor, Cincinnati, OH (US); Jody Lynn Hoying, Maineville, OH (US); Gregory T. Jones, Cincinnati, OH (US); Samuel James St. John, Cincinnati, OH (US)

(21) Appl. No.: 17/308,086

(22) Filed: May 5, 2021

Related U.S. Application Data
(60) Provisional application No. 63/020,036, filed on May 5, 2020.
(60) Provisional application No. 63/020,037, filed on May 5, 2020.

(51) Int. Cl. A61K 8/27; A61K 8/19; A61K 8/25; A61Q 11/00; A61K 8/21; A61K 8/365; A61K 8/362; A61K 8/49; A61K 8/44; A61K 8/24

(52) U.S. Cl. CPC A61K 8/27; A61K 8/19; A61K 8/25; A61Q 11/00; A61K 8/21; A61K 8/365; A61K 2800/28; A61K 8/492; A61K 8/4946; A61K 8/445; A61K 8/44; A61K 8/24; A61K 8/362

(57) ABSTRACT

Methods of use of oral care compositions comprising tin. Methods of increasing the density of teeth, densifying the teeth, and/or reducing the rate of tooth material loss. Remineralizing oral care compositions with tin, silica, chelant, a pH of about 6 or greater, but free of zinc. Remineralizing oral care compositions with tin, silica, monodentate ligand, polydentate ligand, a pH of about 6 or greater, but free of zinc. Remineralizing dentifrice compositions with tin, silica, and a chelant system, but free of zinc.

### FIELD OF THE INVENTION

The present invention is directed to oral care compositions comprising tin. The present invention is also directed to oral care compositions with high amounts of soluble fluoride ions, which can contribute to remineralization of enamel and/or dentin. The present invention is also directed to methods of increasing the density of teeth, enamel, and/or dentin. The present invention is also directed to methods of densifying teeth, enamel, and/or dentin.

### BACKGROUND OF THE INVENTION

Oral care compositions have included antimicrobial agents, such as tin ions, to counter oral bacteria and to prevent and treat conditions caused by bacteria in the oral cavity, such as formation of dental plaque and calculus. The formation of dental plaque and calculus and failure to stop their proliferation are the primary cause of dental caries, gingivitis, periodontal disease, and tooth loss. Additionally, tin ions can deposit on surfaces in the oral cavity to provide protective functions, such as antierosion, antibacterial, and/or antisensitivity benefits.

However, tin can be challenging to properly formulate in oral care compositions due to reactivity between tin and other components of oral care compositions. Under-stabilizing or over-stabilizing tin can lead to lower availability of tin ions to provide the desired benefit. For example, if the tin is under-stabilized, the tin can react with other components of the oral care composition, such as silica, water, etc., which can lead to a lower amount of available tin ions. Additionally, the remaining under-stabilized tin, when delivered to the oral cavity, may be hyper-reactive with different oral surfaces, thus impeding the action of other ingredients, such as fluoride that is key to restore the density of weakened enamel. In contrast, if the tin is over-stabilized or the chelant-tin interaction is too strong, tin ions will be tied up when delivered to the oral cavity, which can also lead to a lower amount of bioavailable tin ions to produce the desired oral care benefit. Thus, the tin-chelant ratio and binding affinity can be carefully balanced to modulate the reactivity of Sn so that it provides its core benefits to the consumer without preventing other active agents in the composition, such as fluoride, from providing their core benefits. As such, there is a need for oral care compositions comprising a high amount of available tin ions that are optimally bioavailable for the desired product benefit.

In particular, dentifrice compositions comprising tin and silica can be challenging to formulate at a pH of about 7 or greater. Tin compounds, such as stannous fluoride, can react with silica abrasive when formulated together in a single phase, which can lead to low levels of soluble fluoride ions. Thus, there is a need for dentifrice compositions comprising tin and silica at a pH of about 7 or greater with sufficient levels of soluble fluoride ions to contribute to remineralization of enamel and/or dentin.

### SUMMARY OF THE INVENTION

Disclosed herein is a method of increasing the density of teeth, densifying teeth, and/or reducing the rate of tooth loss comprising (a) instructing a user to apply a dentifrice composition to a toothbrush, and (b) instructing the user to apply the dentifrice composition to an oral cavity of the user, wherein the dentifrice composition comprises: (i) tin; (ii) abrasive; (iii) monodentate ligand; and (iv) polydentate ligand.

Also disclosed herein is a dentifrice composition comprising (a) tin; (b) abrasive; (c) chelant; and (d) a pH of at least about 6, wherein the dentifrice composition comprises less than 0.01%, by weight of the dentifrice composition, of zinc.

Also disclosed herein is a dentifrice composition comprising (a) tin; (b) abrasive; (c) monodentate ligand; (d) polydentate ligand; and (e) a pH of at least about 6, wherein the dentifrice composition comprises less than 0.01%, by weight of the dentifrice composition, of zinc.

### BRIEF DESCRIPTION OF THE DRAWINGS

FIG. 1 shows an individual exhibiting a loss of tooth density and/or tooth material over the course of a lifetime.

FIGS. 2(A and B) shows and individual exhibiting a loos of tooth density and/or tooth material over the course of a lifetime without the use of the disclosed composition.

FIGS. 3(A and B) shows the impact of the use of the disclosed compositions on mitigating the loss of tooth density and/or tooth material over the course of a lifetime.

### DETAILED DESCRIPTION OF THE INVENTION

The present invention is directed to Sn-containing oral care compositions that have been optimally stabilized for delivering a high amount of bioavailable Sn to the enamel surface while optimizing the rest of the formula components to ensure high fluoride activity in the diluted composition, which reflects the bioavailability of soluble fluoride. High bioavailability of soluble fluoride is important for anti-caries efficacy. The resulting invention also provides compositions without added Zn thereby limiting the formation of a Zn-chelate-F complex. Zn citrate is conventionally used as an anti-tartar agent, and its impact on fluoride activity was not to our knowledge previously reported in the literature. The withholding of added Zn from the composition, therefore, unexpectedly increased the composition fluoride activity relative to the Zn-containing composition.

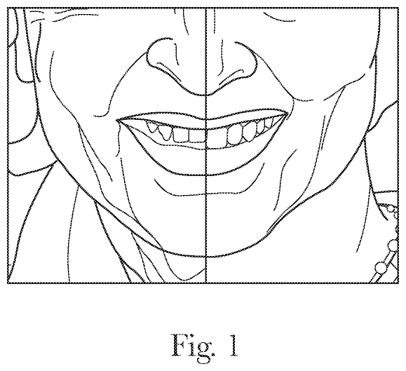

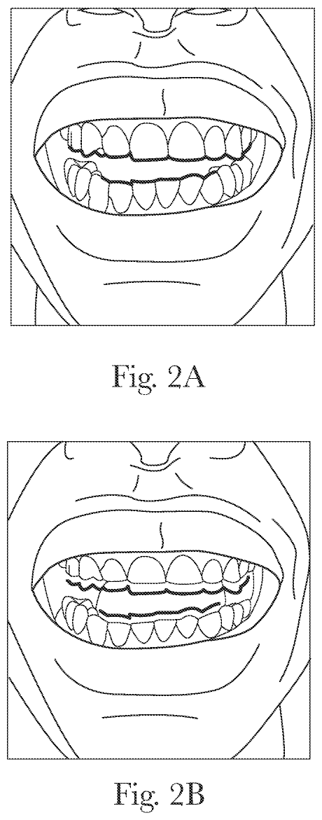

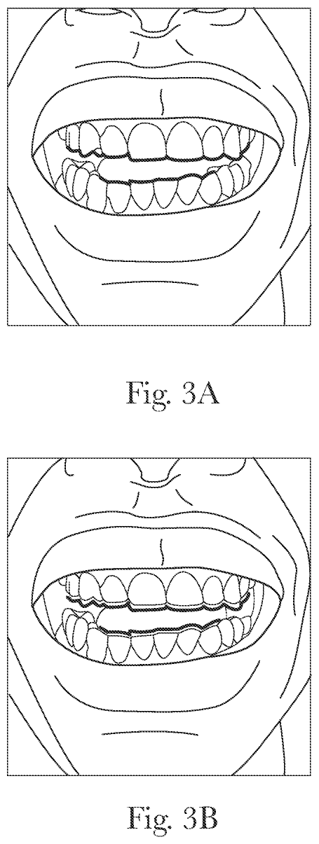

The present invention is directed to methods of increasing the density of teeth, enamel, and/or dentin through the application of the disclosed oral care compositions that optimally deliver a high amount of bioavailable Sn and have high enough fluoride activity. Over the course of a person's life, tooth material, such as enamel and dentin, is lost through chemical and/or physical insults. As a result, and as shown in FIG. 1, as a person ages, the teeth get smaller at a rate of 40 microns/year. The present invention is directed to methods for increasing the density of teeth and/or reducing the rate of tooth material loss, as shown in the comparison between FIG. 2 and FIG. 3. Twice daily use of the disclosed compositions can reduce the amount of tooth material loss and increase the density of teeth, enamel, and/or dentin.

Enamel and dentin are composed of hydroxyapatite mineral. This mineral, through the process of chemical acid and physical degradation, can lose density and/or tooth material over time. When the source of those acids is the biological metabolization of fermentable carbohydrates by bacteria, the end result is density loss leading to a cavity. Fluoride in the composition is responsible for restoring mineral density loss to enamel or dentin. When the sources of those acids are dietary acids, the end result is density loss leading to enamel or dentin erosion. Acids that damage the tooth surface can amplify the physical loss of enamel or dentin through abrasion, abfraction, or attrition. Thus, enamel and/or dentin density loss can be prevented and/or restored by mitigating the damage attributable by acids of all sources.

Many agents can be used protect enamel or dentin from acids. However, the method of application of these agents to the enamel or dentin can strongly impact their efficacy. It has been recently discovered that certain stabilizing agents, which can be used to optimally stabilize Sn leading to optimal Sn reactivity in both the tube or bottle and the oral cavity, can also reduce the efficacy of fluoride through the formation of complexes including fluoride, one or more stabilizing agents, and/or other metal ions, such as zinc. Therefore, unexpectedly, by both optimizing the Sn reactivity and fluoride reactivity, compositions have been discovered that can simultaneously provide high levels of density restoration and density preservation. Thus, it is now possible to deliver this long felt, but unmet need in the marketplace and provide a new oral health benefit to consumers.

While not wishing to being bound by theory, it is believed that by balancing enamel surface protection, such as through effective chelation of stannous in the tube, and remineralization, such as through improved fluoride ion availability, the disclosed compositions can lead to increased teeth density, densifying teeth, and/or the reduction of the rate of tooth loss.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival -paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen’ or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The oral care composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the oral care composition can be incorporated into a film, a strip, a foam, or a fiber-based dentifrice composition.

The oral care compositions, as described herein, comprise tin, monodentate ligand, and polydentate ligand. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, for example, oxalic acid, or salts thereof, can be a dicarboxylic acid, a polydentate ligand, and/or a whitening agent.

### Tin

The oral care composition of the present invention comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. The tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source.

### Monodentate Ligand

The oral care composition can comprise a monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 0.01% to about 10%, from about 0.1% to about 15%, from about 1% to about 5%, or from about 0.0001 to about 25%, by weight of the composition, of the monodentate ligand.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e. with two functional groups), tridentate (i.e. with three functional groups), tetradentate (i.e. with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, citric acid, phytic acid, pyrophosphate, tripolyphosphate, tetrapolyphosphate, hexametaphoshate, salts thereof, and/or combinations thereof.

The oral care composition can include from about 0.01% to about 10%, from about 0.1% to about 15%, from about 1% to about 5%, or from about 0.0001 to about 25%, by weight of the composition, of the polydentate ligand.

### Ratio of Tin to Monodentate Ligand to Polydentate Ligand

The oral care composition, as described herein, can comprise a ratio of tin to monodentate ligand to polydentate ligand that provides an unexpectedly high amount of soluble tin and/or a superior fluoride uptake. Suitable ratios of tin to monodentate ligand to polydentate ligand can be from about 1:0.5:0.5 to about 1:5:5, from about 1:0.5:0.75 to about 1:5:5, from about 1:1:1 to about 1:5:5, from about 1:1:0.5 to about 1:2.5:2.5, from about 1:1:1 to about 1:2:2, from about 1:0.5:0.5 to about 1:3:1, or from about 1:0.5:0.5 to about 1:1:3.

Desired herein are oral care compositions with a soluble Sn of at least about 1000 ppm, 2000 ppm, 4000 ppm, at least about 4500 ppm, at least about 5000 ppm, at least about 6000 ppm, and/or at least about 8000 ppm. Also desired herein are oral care compositions with a fluoride uptake of at least about 6.5 μg/cm², at least about 7.0 μg/cm², at least about 8.0 μg/cm², or at least about 9.0 μg/cm² after a time period of at least about 9 days, 30 days, 65 days, 75 days, 100 days, 200 days, 365 days and/or 400 days.

In total, while not wishing to be bound by theory it is believed that the soluble Sn amount is correlated to bioavailable Sn as it is freely available to provide an oral health benefit. Fully bound Sn (i.e. Sn that is overchelated) or precipitated Sn (i.e. insoluble tin salts, such as Sn(OH)2 and/or Sn-based stains can form when Sn is underchelated) would not be included in the measurement for soluble Sn. Additionally, while not wishing to be bound by theory, it is believed that a carefully balanced ratio of Sn to monodentate and polydentate ligands can provide a high amount of bioavailable fluoride and Sn ions without some of the negatives to the use of cationic antimicrobial agents, such as surface staining. Thus, additional screening experiments were done to quantify and qualify the ranges and identities of monodentate and polydentate ligands.

### Dicarboxylic Acid

The polydentate ligand can comprise dicarboxylic acid. The dicarboxylic acid comprises a compound with two carboxylic acid functional groups. The dicarboxylic acid can comprise a compound or salt thereof defined by Formula I.

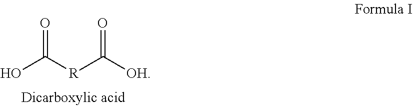

R can be null, alkyl, alkenyl, allyl, phenyl, benzyl, aliphatic, aromatic, polyethylene glycol, polymer, O, N, P, and/or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.01% to about 10%, from about 0.1% to about 15%, from about 1% to about 5%, or from about 0.0001 to about 25%, by weight of the oral care composition, of dicarboxylic acid.

### Tricarboxylic Acid

The polydentate ligand can comprise tricarboxylic acid. The tricarboxylic acid comprises a compound with three carboxylic acid functional groups. The tricarboxylic acid can comprise a compound or salt thereof defined by Formula II.

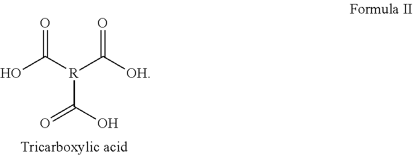

R can be alkyl, alkenyl, allyl, phenyl, benzyl, aliphatic, aromatic, polyethylene glycol, polymer, O, N, P, and/or combinations thereof.

The tricarboxylic acid can comprise citric acid, isocitric acid, aconitic acid, propane-1,2,3-tricarboxcylic acid, trimesic acid, any tricarboxylic acid in the citric acid cycle or Krebs Cycle, salts thereof, or combinations thereof. The tricarboxylic acid can comprise suitable salts of tricarboxylic acid, such as for example, sodium citrate.

The oral care composition can comprise from about 0.01% to about 10%, from about 0.1% to about 15%, from about 1% to about 5%, or from about 0.0001 to about 25%, by weight of the oral care composition, of tricarboxylic acid.

### Polyphosphate

The polydentate ligand can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates, such as phytic acid, of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21, from about 2 to about 14, or from about 2 to about 7. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate. The oral care composition can be essentially free of, substantially free of, or free of cyclic polyphosphate. The oral care composition can be essentially free of, substantially free of, or free of phytic acid.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.5% to about 1.5%, or from about 0.3% to about 0.6%, by weight of the dentifrice composition. In particular, zinc can be detrimental to the remineralization process and/or lead to complexes with fluoride and certain ligands that can limit the fluoride efficacy. Thus, the oral care composition can be essentially free of, substantially free of, or free of zinc.

pH

The pH of the oral care compositions as described herein can be from about 4 to about 7.5, from about 4.5 to about 6.5, or from about 4.5 to about 5.5. The pH of the oral care compositions, as described herein, can also be at least about 6, at least about 6.5, or at least about 7. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water. The pH of the oral care compositions as described herein have a preferred pH of from about 4 to about 10, from about 5 to about 9, from about 6 to 8, or about 7.

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

### Amino Acid

The oral care composition can comprise amino acid. The monodentate and/or polydentate ligand can comprise amino acid. Whether the amino acid is a monodentate ligand or polydentate ligand can be based on how many functional groups capable of chelating to, associating with, and/or bonding to tin are present and/or the pH of the oral care composition. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Suitable amino acids include the compounds described by Formula III, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

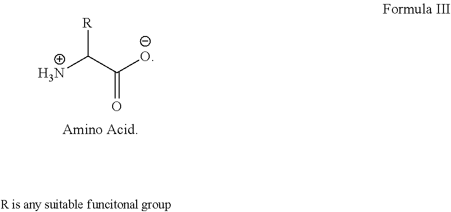

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acid which has an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the tin, monodentate ligand, and/or polydentate ligand include emulsion compositions, such as the emulsions compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Methods

The oral care compositions, as described herein, can lead to oral health benefits, such as the remineralization of teeth, when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure. The user can also be instructed to rinse with a mouthwash composition comprising a therapeutic amount of fluoride and/or mouth rinse composition comprising a therapeutic amount of fluoride after the completion of the brush procedure. The user can also be instructed to not rinse with any liquid, including tap or bottled water, other than a composition comprising a therapeutic amount of fluoride. As the application of the oral care composition can lead to oral health benefits, such as the remineralization of teeth, rinsing the oral cavity after application and expectoration of the oral care composition can remove residual fluoride from the surface of teeth, thereby at least partially diminishing the oral health benefit.

Other oral health benefits that can result from the use of the oral care composition in an oral cavity, such as in the application of the oral care composition to teeth, include increasing the density of teeth, the prevention of the loss of calcium from the teeth, repairing structural weaknesses in enamel, extending the life of a user's teeth, increasing the structural density of enamel, coating enamel with rebuilding minerals, and/or remineralization of teeth.

Disclosed herein are methods for increasing the density of teeth, the prevention of the loss of calcium from the teeth, repairing structural weaknesses in enamel, extending the life of a user's teeth, increasing the structural density of enamel, coating enamel with rebuilding minerals, and/or remineralization of teeth comprising instructing a user to apply an oral care composition, as described herein, for at least 1 minute twice a day. The method can also include instructing a user to expectorate the oral care composition and either not rinsing the oral cavity or only rinsing the oral cavity with a composition comprising a therapeutic amount of fluoride.

Also disclosed herein are methods to densify teeth. As described herein, “densify” means that the disclosed compositions can provide (i) surface protection through effective chelation of stannous ions after manufacture, but before use; and (ii) remineralization through improved fluoride ion availability, such as through the removal of zinc or other competing metal ions.

While not wishing to being bound by theory, it is believed that use of the disclosed compositions over the course of 1 month, 6 months, 1 year, 5 years, 10 years, 15 years, 20 years, 25 years, 30 years, and/or an entire lifetime can lead to teeth with an increased density and/or a reduction of the loss of tooth material that typically happens during ordinary daily use, such as in the left side of FIG. 1. Additionally, FIG. 2 shows the impacts of the loss of tooth material over the course of a lifetime. FIG. 2A is the before image and FIG. 2B is the after image, which displays the loss of tooth material over the course of a lifetime without the use of the disclosed compositions. FIG. 3 shows the potential impact of use of the disclosed compositions in reducing the loss of tooth material over the course of a lifetime. FIG. 3A is the before image and FIG. 3B is the after image, which displays the loss of tooth material over the course of a lifetime with the use of the disclosed compositions.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Compositions

TABLE 1
Compositions

| | Example 1 | Example 2 |
|---|---|---|
| Component | (wt %) | (wt %) |
| Glycerin | — | 27.013 |
| Sorbitol | 48.000 | 34.900 |
| Treated Water | 21.581 | 8.186 |
| SnF₂ | 0.454 | 0.454 |
| SnCl₂ 10% silica blend | 0.562 | 0.440 |
| Sodium Gluconate | 1.300 | 1.020 |
| NaOH (50%) | 0.870 | 0.720 |
| Saccharin | 0.400 | 0.5000 |
| Sucralose (25%) | 0.200 | 0.1600 |
| Xanthan Gum | 0.875 | 0.6125 |
| Carrageenan | 1.500 | 1.050 |
| Zinc Citrate | 0.533 | — |
| Na Citrate | — | 1.220 |
| TiO₂ | 0.500 | 0.5000 |
| Silica | 17.500 | 15.000 |
| SLSS (29%) | 5.000 | 5.625 |
| Cocamidopropyl Betaine (30%) | | 1.500 |
| Flavor | 1.175 | 1.100 |

TABLE 1 provides the compositions described herein. Example 1 includes stannous fluoride, silica abrasive, and zinc citrate. Example 2 includes stannous fluoride, silica abrasive, but is free of all zinc salts. Once made, the example compositions were allowed to age approximately 6 months at room temperature. The soluble fluoride activity relative to that of Crest® Cavity Protection (Procter & Gamble, Cincinnati, Ohio, USA) at its native pH (about pH 7) in each composition was determined in TABLE 8 under a variety of pH conditions.

### Determination of Slurry Fluoride Content

Approximately 15 g of toothpaste was placed into a 60 mL syringe careful to exclude any air from the syringe. In a second syringe, 45 g of ultrapure, 18 MΩ (DI) water was portioned careful to exclude any air from the syringe. The two syringes were locked together via a Luer lock connector and alternately plunged back and forth until the composition was completely mixed at least 20 back-and-forth plunges in about 2 minutes. The resulting slurry was expelled from the syringes into a beaker containing a cross-shaped stir bar.

The resulting slurry was placed on a magnetic stir plate mixer and a pH and fluoride ion selective electrode (Thermo Scientific, Orion, 96-09-00, Waltham, Mass.) were placed into the slurry. Both electrodes had been previously calibrated according to the manufacturer's instructions. A calibration curve was developed for the fluoride ion selective electrode using a fluoride standard diluted 1:1 with TISAB II (Sigma Aldrich, Merck KGaA, Darmstadt, Germany). The stir plate was switched on and a speed was selected to ensure vigorous mixing. Simultaneous measurements of pH and fluoride were obtained as the pH was manipulated dropwise with 1N HCl or 1N NaOH while ensuring both electrodes had stabilized before recording their values. Once the acid or base was added, the stir plate was switched off to allow the electrodes to stabilize. The measurement was recorded and the procedure was repeated to create the fluoride-activity curve of each toothpaste.

### Artificial Caries Lesion Remineralization Method

Increases in the density of oral hard tissues were quantified using this Lesion Remineralization Method (LRM).

Caries free human teeth (erupted third molars, molars, and pre-molars) were inspected under a stereomicroscope (Leica M80, Leica Microsystems Inc., Buffalo Grove, Ill.) on the buccal and lingual surfaces for suitable crack-free windows (about 4×4 mm). Suitable windows were marked with a pencil and these specimens were saved for coring. Specimens were prepared by cutting enamel cores from the collected teeth in the suitable crack-free window using a diamond core drill. Each specimen was mounted in a ¼ inch diameter Lucite rod using dental acrylic (Durabase, Reliance Manufacturing Company, Worth, Ill., USA) covering all sides except the natural facial surface. Specimens were polished with 600 grit silicon carbide-water slurry to remove approximately 50 μm of the outer enamel. Specimens were then polished for an additional 90 minutes with gamma alumina (Linde No. 3, AB Gamma Polishing Alumina, Buehler Limited, Lake Bluff, Ill., USA). Any specimen found to have visible surface imperfections were rejected. Samples were then prepared generally as below:
1) A fluoride pretreatment to condition the freshly polished surface;
2) The creation of an artificial caries lesion;
3) The mounting of a porous, diffusion-control film;
4) The remineralization of the artificial caries lesion; and finally,
5) The analysis of the remineralized artificial caries lesion.

In summary, prepared human enamel rod specimens were pretreated with a fluoride presoak for 24 hrs then exposed to a demineralization solution for 36 hours to create a lesion. The specimens were then subjected to a cycling regimen for 30 days, each day consisted of a first dentifrice treatment followed by soaking in a remineralizing solution then as second dentifrice treatment. Samples were left overnight in a reamizeralizing solution. At the end of cycling, the lesions were sagittally cross sectioned, embedded in resin, polished, and analyzed for mineral content. Effectiveness was determined by comparing the amount of enamel remineralization relative to a non-fluoride/silica toothpaste or to a 1100 ppm fluoride as NaF/silica toothpaste. Approximately 24 specimens were used for each treatment group.

The following treatments and reagents were used in the study:

### Experimental Treatments

Dentifrice products were treated as a 1:3 (paste:water) slurry. The slurry was formed by homogenizing for one minute the paste with ultra pure water in an appropriate mixer to ensure uniformity.

TABLE 2
Sodium Fluoride Stock Solution (1300 ppm)

| | | | | Target |
|---|---|---|---|---|
| | | Molecular | Target | Weight per |
| Raw Materials | Formula | Weight | ppm | Liter (g) |
| Sodium Fluoride, Anhydrous | NaF | 41.99 | 1300 | 2.875 |
| Deionized Water | H₂O | — | — | QS to 1000 |

The sodium fluoride stock solution of TABLE 2 was made by adding first water to a beaker witholding 10% of the final volume required, adding sodium fluoride as indicated in TABLE 2 and stirring until completley mixed, then by trasnfering to a volumetric flask and adding the remaining water to reach 1000 mL.

TABLE 3
Sodium Phosphate Stock Solution (200 mM)

| | | | | Target |
|---|---|---|---|---|
| | | Molecular | Target | Weight per |
| Raw Materials | Formula | Weight | Molarity | Liter (g) |
| Sodium Phosphate, | NaH₂PO₄*H₂O | 137.99 | 0.2 | 27.60 |
| Monobasic | | | | |
| Deionized Water | H₂O | — | — | QS to 1000 |

The sodium phosphate stock solution of TABLE 3 was made by adding first water to a beaker witholding 10% of the final volume required, adding sodium phosphate as indicated in TABLE 3 and stirring until completley mixed, then by trasnfering to a volumetric flask and adding the remaining water to reach 1000 mL.

TABLE 4
Calcium Chloride Stock Solution (200 mM)

| | | | | Target |
|---|---|---|---|---|
| | | Molecular | Target | Weight per |
| Raw Materials | Formula | Weight | Molarity | Liter (g) |
| Calcium Chloride, | CaCl* | 147.02 | 0.2 | 29.40 |
| Dihydrate | 2H₂O | | | |
| Deionized Water | H₂O | — | — | QS to 1000 |

The calcium chloride stock solution of TABLE 4 was made by adding first water to a beaker witholding 10% of the final volume required, adding calcium chloride as indicated in TABLE 4 and stirring until completley mixed, then by trasnfering to a volumetric flask and adding the remaining water to reach 1000 mL.

TABLE 5
Fluoride Presoak Solution

| | | | | Target | Target |
|---|---|---|---|---|---|
| | | | | Weight | Volume |
| | | | | per | per |
| | | Molecular | Target | Liter | Liter |
| Raw Materials | Formula | Weight | Molarity | (g) | (ml) |
| 200 mM | NaH₂PO₄*H₂O | 137.99 | 0.001 | — | 5.00 |
| Sodium | | | | | |
| Phosphate | | | | | |
| Stock Solution | | | | | |
| 200 mM | CaCl₂*2H₂O | 147.02 | 0.001 | — | 5.00 |
| Calcium | | | | | |
| Chloride | | | | | |
| Stock Solution | | | | | |
| Sodium | NaF | 41.99 | 0.001 | 0.0420 | — |
| Fluoride, | | | | | |
| Anhydrous | | | | | |
| Sodium | NaCl | 58.44 | 0.0361 | 2.1097 | — |
| Chloride, | | | | | |
| Anhydrous | | | | | |
| Sodium | HCl | 36.46 | — | — | — |
| Hydroxide, | | | | | |
| 0.1N | | | | | |
| Deionized | H₂O | — | — | — | QS to |
| Water | | | | | 1000 |

The samples were incubated overnight in the fluoride stock solution prior to the start of lesion creation to prevent excessive erosion to the surface of the specimen. The fluoride presoak stock solution of TABLE 5 was made by adding first water to a beaker witholding 20% of the final volume required, adding sodium phosphate stock solution as indicated in TABLE 5 and stirring until completley mixed, then by adding the calcium chloride stock solution as indicated in TABLE 5, then by adding sodum fluoride as indicated in TABLE 5 and stirring until completely mixed, and then by adding sodium chloride as indicated in TABLE 5 and stirring until completely mixed. The pH was adjusted to 5.1 using sodium hydroxide as indicated in TABLE 5, then the solution was transferred to a volumetric flask and the remaining water was added to reach 1000 mL. Before each use, the pH was remeasured and adjusted to pH 5.1 as needed.

TABLE 6
Demineralization Solution for Lesion Formation

| | | | | | | Target | Target |
|---|---|---|---|---|---|---|---|
| | | | | | | Weight | Volume |
| | | Molecular | Target | | Mass | per Liter | per Liter |
| Raw Materials | Formula | Weight | Molarity | Density | % | (g) | (ml) |
| Glacial Acetic | CH₃COOH | 60.05 | 0.075 | 1.05 | — | 4.5038 | 4.29 |
| Acid | | | | | | | |
| 200 mM Sodium | NaH₂PO₄* | 137.99 | 0.002 | — | — | — | 10.00 |
| Phosphate | H₂O | | | | | | |
| Stock Solution | | | | | | | |
| Sodium Hydroxide, | NaOH | 40.00 | — | — | — | — | 10.00 |
| 2N (Step 4) | | | | | | | |
| 200 mM Calcium | CaCl₂*2H₂O | 147.02 | 0.002 | — | — | — | 10.00 |
| Chloride Stock | | | | | | | |
| Solution | | | | | | | |
| Carbopol 907 | | Polymer | — | — | 0.2 | 2.00 | — |
| Sodium Hydroxide, | NaOH | 40.00 | — | — | — | — | 4.00 |
| 2N (Step 9) | | | | | | | |
| Deionized Water | H₂O | — | — | — | — | — | QS to 1000 |

The demineralizing solution served as an acid challenge similar to that generated by plaque acids. The addition of Carbopol helped protect the ground and polished specimen cores from erosion during lesion formation.

The demineralization solution for lesion formation of TABLE 6 was made by adding first water to a beaker witholding 20% of the final volume required, adding acetic acid as indicated in TABLE 6 and stirring until completley mixed, then by adding the sodium phosphate solution as indicated in TABLE 6, then by adding the Step 4 sodium hydroxide stock solution as indicated in TABLE 6, then by adding the calcium chloride stock solution very slowly (dropwise) with stirring until completely mixed, then by adjusting to pH 4.3. Next, the Carbopol was weighed and added to the solution with stirring. The solution was covered with plastic wrap and was allowed to stir overnight until the Carbopol was completely incorporated into the solution. The next day, the Step 9 sodium hydrixde solution as indicated in TABLE 6 was added dropwise to adjust the pH to 4.3. Finally, the solution was transferred to a volumetric flask and water was added to bring the volume of the solution to 1000 mL. The pH is checked each time before use and is adjusted to pH 4.3.

TABLE 7
Remineralization Solution

| | | | | Target | Target |
|---|---|---|---|---|---|
| | | | | Weight | Volume |
| | | | | per 4 | per 4 |
| | | Molecular | Target | Liters | Liters |
| Raw Materials | Formula | Weight | Molarity | (g) | (ml) |
| Calcium Nitrate, | Ca(NO₃)₂* | 236.15 | 0.0015 | 1.4169 | — |
| Tetrahydrate | 4H₂O | | | | |
| Potassium | KH₂PO₄ | 136.09 | 0.0009 | 0.4899 | — |
| Phosphate | | | | | |
| Potassium | KCl | 74.55 | 0.15 | 44.73 | — |
| Chloride | | | | | |
| BisTris | C₈H₁₉NO₅ | 209.24 | 0.02 | 16.74 | — |
| (CAS 6976-37-0) | | | | | |
| Hydrochloric | HCl | 36.46 | — | — | 9.00 |
| Acid, 2N | | | | | |
| Deionized Water | H₂O | — | — | — | QS to |
| | | | | | 4000 |

The remineralization solution of TABLE 7 was made by adding first water to a beaker witholding 10% of the final volume required, then by adding calcium nitrate as indicated in TABLE 7 and stirring until completely mixed, then by adding potassium phosphate as indicated in TABLE 7 and stirring until completely mixed, then by adding potassium chloride as indicated in TABLE 7 and stirring until completely mixed, then by adding BisTris as indicated in TABLE 7 and stirring until completely mixed. Then hydroxhloric acid was added dropwise to adjust the pH to 7.0 (6.95-7.05). The solution was then transferred to avolumentric flask and water was added to bring the volume to 4000 mL as indicated in TABLE 7. The pH was checked and adjusted to pH 7.0 as necessary before each use.

### Treatment Procedure

Specimens were initially exposed to a fluoride presoak solution to condition the surface of the ground and polished enamel. 10 mL per specimen of fluoride presoak solution was added into a deep-well reservoir. The specimen holder was placed over the reservoir making sure the end of each specimen was submerged in the solution. The specimens were then incubated at 37° C. with gentle shaking for 24 hours. After incubation, the specimens were removed from the fluoride presoak solution and were rinsed with ultra-pure water. The presoak must be completed early enough in the week to allow for the completion of lesion formation, then to equilibrate biofilm, and at least 1 treatment cycle to be completed before the weekend.

Specimens were then exposed to the demineralization solution for lesion formation to create an artificial caries lesion. 10 mL per specimen of demineralization solution was added into a deep-well reservoir. The specimen holder was placed over the reservoir making sure the end of each specimen was submerged in the solution. The specimens were then incubated at 37° C., without agitation, for 36 hours. At the end of the demineralization period, the specimens were rinsed thoroughly with water.

Specimens were then encased in a porous, diffusion-control film. This porous, diffusion-control film was prepared from three layers of material and a plastic shroud that that had a window allowing intimate contact between the specimen surface and the treatment slurries or remineralization solution. The layers were prepared by first hole punching thick chromatography cellulose paper (Grade 238, Ahlstrom, 7×8 cm, VWR, USA) using a 0.25″ hole punch and collecting the resulting circles of paper. Each specimen required 2 cellulose layers to create a porous, diffusion-controlled film that as approximate 700 μm thick. A third layer of cotton gauze (Polyester Rayon Non-Woven Gauze, VWR, USA) was hole punched using 0.25″ hole punch and the resulting circles of gauze were collected. One layer of cotton gauze was needed for each specimen. A plastic shroud was fashioned from a cup sleeve washer cap (Electrical-Insulating Cup Sleeve Washer, hole diameter drilled to 4 mm diameter, McMaster-Carr, USA) that was placed with the smaller hole facing down on a flat surface. One layer of gauze followed by two layers of cellulose were then gently press into the bottom of the cup sleeve washer. A cap/diffusion-controlled media was prepared for each specimen. Once the caps were assembled, one was placed on the end of each specimen rod, covering the enamel end. With the cap facing up, each rod was prewetted with ultra-pure water and allowed to hydrate before placing cap side down in remineralization solution to avoid trapping any bubbles under the cap. If caps were not secure, replace the cap with a mechanically tightly fitted cap. It is critical that the caps were snuggle fitted uniformlly around the specimen to prevent leakage of treatment slurries under the shroud that then directly contacted the enamel surface. The capped specimens were then placed into 200 mL of remineralization solution overnight until treatment cycling began the next morning.

The treatment cycle occurred every 24 hours as indicated below and was repeated for a total of 30 treatment days. The samples were left in quiescent reminalziation solution in a 37° C. incubator over weekend/non-treatment periods.
1) Preparation: For each treatment group, the treatment reservoir, rinse reservoir, and two remineralization reservoirs were labeled. All necessary reservoirs were labelled prior to start. The rinse reservoirs were filled with 100 mL ultra-pure water. The remineralization reservoirs were filled with 200 ml of remineralization solution.
2) Slurry Making: Dentifrice slurries (25% paste in water) were prepared by mixing 1 part dentifrice (15 g) with three parts ultra-pure water (45 g) for one minute until completely homogenized using a non-aerating technique. The total volume of the slurry equaled approximately 60 mLs per treatment (this volume was the minimum necessary to fill the treatment reservoir to an appropriate level). When treating, it was ensured that the specimen surfaces are submerged in the treatment slurry.
3) Morning Treatment: The specimens were immersed in the slurry and agitated on a titer plate shaker (ThermoFisher, USA) at speed 2 for 2 minutes. The slurries were mixed just prior to the treatment and were discarded after use.
4) Rinse: After the 2-minute treatment, the specimens were transferred from the treatment reservoir to the rinse reservoir. The were rinsed by agitating the specimens in the ultra-pure rinse water on the titer plate shaker for 20 seconds. Each treatment group was rinsed in a different rinse reservoir to avoid carryover between treatments. Rinse water was discarded after use.
5) Daytime Remineralization Period: Following the morning treatment and rinse, the specimens were placed into fresh remineralization solution and the used remineralzilation solution was discarded. The specimens were incubated at 37° C., without agitation, for 6 hours.
6) Afternoon Treatment, Rinse: The specimens were treated and rinsed as in steps 3 and 4.
7) Overnight Remineralization Period: After the afternoon treatment and rinse, the specimens were placed back into their daytime remienralization solution and into an incubator at 37° C., without agitation, overnight.
8) Repeat: The next morning, all steps 1-7 were repeated as before. Samples were left in remineralization solution over the weekend.

After cycling, the specimens were carefully separated from their caps and were rinsed thoroughly. Care was taken to not touch the specimen surface. Using a high precision diamond saw, each specimen was then cut in half vertically (from treated surface down through the lesion) through the lesion window. The specimens were mounted as a group together (up to 12 per block) in a 40-millimeter diameter round block with VersoCit 2 cold-set acrylic resin (Struers, Cleveland, Ohio, USA) covering all surfaces except the cut face. Once set and to permit visualization of the surface for cross sectional micro hardness indents, each block was sanded and polished blocks using the Struers Tegramin-30 polisher using 600 grit water-wetted sandpaper, then a series of liquid polishing at 9, 3, and 1 μm DiaPro diamond abrasives according to the manufacturers instructions (Struers, Cleveland, Ohio, USA). After polishing, the blocks are ready to analyze.

Cross-section lesions were indented using the following method. Following polishing, indentations were made with the long axis of the diamond parallel to the outer enamel surface at regular intervals across the lesion and into the underlying sound enamel. A Knoop diamond (Wilson Hardness Tukon 1202, Buehler a division of Illinois Tool Works, Lake Bluff, Ill.) was used under a 10- or 50-gram load. The 10-gram load was used to make the first indent 13 microns from the surface of the tooth. Additional indents were made through the body of the lesion at 13-micron increments yielding a total of 7, 10-gram-load indents in a line. The 50-gram load was used to make indents 25 microns from the last 10-gram-load indent and at 25-micron intervals for a total of 8, 50-gram-load indents in the sound enamel. This process was repeated, such that each sample had two lines of indents to assess the average hardness through the body of the lesion. The Knoop hardness number (KHN) was converted into volume percent mineral (vol % mineral) using Equation 1.

(KHN)^{1/2}=0.197 (vol % mineral)−0.24 Equation 1

The vol % mineral lost (mineral loss) was calculated as the area between the total integrated area and the integrated area from the normalized volume percent mineral values from the measurement points. The total integrated area corresponds to the range of the measurement points in units of microns times the average volume percent mineral value determined for the sound enamel region. The area calculation used the trapezoidal rule. The mean mineral loss for the treatment group was obtained by averaging each specimen's mineral loss within a treatment group.

Microscope images were also obtained under reflected brightfield illumination at 5× magnification using a Nikon Optiphot-2 microscope (Nikon, Japan) outfitted with a Moticam 2300 (Motic America, Richmond, British Columbia, Canada) to record digital images. Images were changed to greyscale and adjusted so the pixel lightness range was 0-255. A region of the image (100×250 pixels) through the body of a representative portion of the lesion was converted to vol % mineral by interpolating the lightness value (0 vol % mineral=0 pixel lightness, 87 vol % mineral=255 pixel lightness). Pixel length was calibrated using the lengths of indents obtained during hardness measurements. Lesion profiles were integrated to obtain the mineral loss and compared for each treatment condition.

$$Remineralization ( \% ) = ( \frac{Mineral {Loss}_{Placebo} - Mineral {Loss}_{Test Group}}{Mineral {Loss}_{Placebo}} ) \times 100 \qquad Equation II$$

### Erosion Cycling Method

The enamel loss observed during erosion cycling according to TABLE 2 was determined by an in vitro model that evaluated the relative ability of oral care compositions to protect tooth surfaces against both the initiation and progression of erosive acid challenges. This model is correlated to predict clinical outcomes in an in-situ model. Briefly, tooth specimens, in groups of five per test, were cycled through 20 treatment cycles over 5 days (4 per day). Each treatment cycled progressed according to the following:
1) Two-minute exposure to a toothpaste slurry;
2) Rinsing with copious amounts of deionized water;
3) 60-minute exposure to freshly collected, whole, human saliva;
4) 10-minute erosive challenge in a solution of 1% (w/q) citric acid;
5) Rinsing with copious amounts of deionized water;
6) 60-minute exposure to freshly collected, whole, human saliva.
   Samples were stored in human saliva in a refrigerator overnight.

The erosion cycling method used here is described in detail by Eversole et al., Erosion Prevention Potential of an Over-the-Counter Stabilized SnF₂ Dentifrice Compared to 5000 ppm F Prescription-Strength Products. J. Clin. Dent. 26 (2015) 44-49, which is herein incorporated by reference.

The only change to the method was an increase in the number of enamel samples and that an optical profilometer (ContourGT 3D Optical Microscope, Bruker USA, Tucson, Ariz., USA) was used to measure the 3d surface topography of the eroded samples. The average eroded depth was determined by integrating the volume of the void caused by the acid erosion with respect to the uneroded, masked reference surface and dividing it by the area of the acid-exposed enamel. Analysis of 3d measurements was done in TalyMap 3D (Taylor Hobson USA, West Chicago, Ill., USA).

Crest Cavity Protection (1100 ppm F as NaF, Procter & Gamble, Cincinnati, Ohio, USA) and Crest ProHealth Advanced Deep Clean Mint (1100 ppm F as SnF₂, Procter & Gamble, Cincinnati, Ohio, USA) were used as the negative and positive controls respectively. The results of the test are only valid if difference in the enamel loss of the negative and positive controls is greater than 25% the value of the enamel loss of the negative control according to Formula III. The test should be repeated if this condition is not met.

$$Erosion Reduction ( \% ) = ( \frac{Enamel {Loss}_{Neg Ctrl} - Enamel {Loss}_{Pos Ctrl}}{Enamel {Loss}_{Neg Ctrl}} ) \times 100 \qquad Equation III$$

TABLE 8
% Fluoride Released vs. Positive Control

| | | Example 1 | | Example 2 | |
|---|---|---|---|---|---|
| USP SnF₂ | | (with Zn) | | (without Zn) | |
| | % F | | % F | | % F |
| | Released vs. | | Released | | Released |
| | Positive | | vs. Positive | | vs. Positive |
| pH | Control | pH | Control | pH | Control |
| 8.34 | 107.5 | 7.00 | 70.5 | 7.99 | 119.2 |
| 7.75 | 107.1 | 6.60 | 69.0 | 7.94 | 117.9 |
| 7.18 | 105.4 | 6.25 | 70.5 | 7.26 | 112.2 |
| 6.65 | 102.7 | 5.90 | 68.5 | 6.44 | 110.0 |
| 6.15 | 96.2 | 5.55 | 71.8 | 5.99 | 106.3 |
| 5.38 | 89.1 | 5.25 | 69.0 | 5.62 | 103.1 |
| 4.99 | 82.6 | 5.05 | 67.3 | 5.29 | 98.1 |
| 4.62 | 71.7 | 4.95 | 63.9 | 5.03 | 92.6 |
| 4.42 | 62.9 | 4.65 | 54.7 | 4.8 | 87.5 |
| 4.36 | 60.6 | 4.3 | 50.4 | 4.6 | 78.9 |
| 4.07 | 46.2 | 4.1 | 43.3 | 4.38 | 67.9 |

TABLE 8 shows the soluble fluoride activity under a variety of pH conditions relative to that of the Crest Cavity Protection slurry at its native pH (100% at approximately pH 7). Additionally, over all pH ranges, Example 2 (free of Zn) demonstrated higher fluoride activity in the toothpaste slurry than Example 1 (including zinc citrate).

TABLE 9
Remineralization and Erosion Reduction

| | Anti- | | % Remin | % Erosion |
|---|---|---|---|---|
| | Caries | Anti- | v CCP, | Red v CCP, |
| Treatment | Agent | Tartar Agent | 0 ppm F | 1100 ppm F |
| CCP, 0 ppm F | None | None | N/A | −12.6 |
| Crest Cavity Protection, | NaF | None | 45.9% | N/A |
| 1100 ppm F | | | | |
| Crest Cavity Protection, | NaF | None | 73.0% | 12.0% |
| 5000 ppm F | | | | |
| Crest Tartar Control, | NaF | Na- | 17.7% | 12.5% |
| 1100 ppm F | | Pyrophosphate | | |
| Example 1, 1100 ppm F | SnF2 | Zn-Citrate | 36.5% | 30.3% |
| Example 2, 1100 ppm F | SnF2 | None | 48.4% | 35.5% |

The % Remin results in TABLE 9 illustrate that as fluoride content was increased in the NaF-containing products in the absence of an agent that interferes with fluoride or tartar formation, the amount of mineral restored to the enamel increases. It is believed that this is a direct result of increasing the mineral content and/or mineral density of the enamel substrate. There is no difference in the performance of SnF₂ to NaF in the absence of an anti-tartar agent for % Remin density increase (Example 2 compared to Crest Cavity Protection, 1100 ppm F). While not wishing to be bound by theory, it is believed to be due to the reactivity of Sn has been optimized without interfering with fluoride's mechanism of action to increase the density of weakened enamel whilst simultaneously removing the Zn citrate to increase the availability of fluoride. The remineralization benefit is reduced and less density is restored to the enamel in either the presence of (i) Zn-Citrate (a compound that can form a complex with fluoride); or (ii) Na-Pyrophosphate (a compound that can modulate calcium degree of saturation).

The % Erosion Reduction results in TABLE 9 illustrate that as one increases fluoride content in the absence of an agent that interferes with fluoride or tartar formation the amount of erosion reduction is increased, but only slightly. Fluoride on its own is a poor agent to prevent the density loss of mineral to erosive acids (dietary acids). Furthermore, anti-tartar agents like Zn-Citrate and Na-Pyrophosphate are known to help reduce erosion. It was unexpected, therefore, that by removing the anti-tartar agent that we were able to simultaneously increase the % Remin and not sacrifice any of the % Erosion Reduction. As the results in TABLE 9 illustrate, the composition of Example 2 can increase the enamel density with a % Remin value comparable to the Crest Cavity Protection, 1100 ppm F while also protecting enamel density and providing a high amount of % Erosion Reduction. While not wishing to be bound by theory, it is believed that through proper Sn stabilization in Example 2, we have achieved a Sn species that is not underchelated such that it binds to the enamel surface preventing remineralization and, simultaneously, not overchelated preventing its reaction with enamel to prevent erosion. This was not possible in the case of Example 1 where non-optimally stabilized Sn and in the presence of Zn citrate was not able to deliver high levels of both % Remin and % Erosion Reduction. By optimally modulating the reactivity of Sn in the SnF₂ anti-caries agent, an oral care composition is provided that is simultaneously capable of increasing the density of the tooth through remineralization while preventing density lost from protecting against erosion. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of increasing the density of teeth, densifying teeth, and/or reducing the rate of tooth loss comprising:
(a) instructing a user to apply a dentifrice composition to a toothbrush, and
(b) instructing the user to apply the dentifrice composition to an oral cavity of the user,
wherein the dentifrice composition comprises:
(i) tin;
(ii) abrasive; and
(iii) monodentate ligand, polydentate ligand, or combinations thereof

2. The method of claim 1, wherein the dentifrice composition comprises a pH of at least about 6.

3. The method of claim 1, wherein the dentifrice composition comprises less than 0.01%, by weight of the dentifrice composition, of zinc.

4. The method of claim 3, wherein the dentifrice composition is essentially free of zinc.

5. The method of claim 3, wherein the dentifrice composition is substantially free of zinc.

6. The method of claim 1, wherein the method further comprises:
(c) instructing the user to not swallow the dentifrice composition.

7. The method of claim 1, wherein the tin comprises stannous fluoride, stannous chloride, or combinations thereof.

8. The method of claim 1, wherein the abrasive comprises silica abrasive, calcium abrasive, or combinations thereof.

9. The method of claim 1, wherein the dentifrice composition has a tin to monodentate ligand to polydentate molar ratio of from about 1:0.5:0.5 to about 1:5:5.

10. The method of claim 9, wherein the molar ratio of tin to monodentate ligand to polydentate ligand is from about 1:1:0.5 to about 1:2.5:2.5.

11. The method of claim 10, wherein the molar ratio of tin to monodentate ligand to polydentate ligand is from about 1:1:1 to about 1:2.5:2.5.

12. The method of claim 1, wherein the dentifrice composition comprises fluoride.

13. The method of claim 13, wherein the fluoride comprises stannous fluoride, sodium fluoride, sodium monofluorophosphate, amine fluoride, or combinations thereof.

14. The method of claim 1, wherein the dentifrice composition comprises no added water.

15. The method of claim 1, wherein the dentifrice composition comprises water.

16. The method of claim 1, wherein the dentifrice composition comprises up to 45%, by weight of the composition, of water.

17. The method of claim 1, wherein the monodentate ligand comprises a compound comprising a single functional group capable of chelating tin.

18. The method of claim 17, wherein the monodentate ligand comprises carboxylic acid or a salt thereof.

19. The method of claim 18, wherein the carboxylic acid comprises aliphatic carboxylic acid, aromatic carboxylic acid, sugar acid, salts thereof, or combinations thereof.

20. The method of claim 19, wherein the sugar acid comprises aldonic acid, ulsonic acid, uronic acid, aldaric acid, salts thereof, or combinations thereof.

21. The method of claim 19, wherein the sugar acid comprises gluconate.

22. The method of claim 19, wherein the aliphatic carboxylic acid comprises linear saturated carboxylic acid, linear unsaturated carboxylic acid, alpha hydroxy acid, beta hydroxy acid, gamma hydroxy acid, amino acid, salts thereof, or combinations thereof.

23. The method of claim 22, wherein the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

24. The method of claim 22, wherein the alpha hydroxy acid comprises lactate.

25. The method of claim 1, wherein the polydentate ligand comprises a compound comprising at least two functional groups capable of chelating tin.

26. The method of claim 25, wherein the polydentate ligand comprises carboxylic acid, polyphosphate, salts thereof, or combinations thereof.

27. The method of claim 26, wherein the carboxylic acid comprises dicarboxylic acid, tricarboxylic acid, salts thereof, or combinations thereof.

28. The method of claim 27, wherein the dicarboxylic acid comprises a compound with the formula HO₂C—R—CO₂H.

29. The method of claim 28, wherein R is aliphatic, aromatic, or combinations thereof.

30. The method of claim 27, wherein the dicarboxylic acid comprises oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, salts thereof, or combinations thereof.

31. The method of claim 27, wherein the tricarboxylic acid comprises citric acid, isocictric acid, aconitic acid, propane-1,2,3-tricarboxylic acid, trimesic acid, salts thereof, or combinations thereof.

32. The method of claim 26, wherein the polyphosphate comprises pyrophosphate, tripolyphosphate, tetrapolyphosphate, hexametaphosphate, or combinations thereof.
